(19) United States
(12) Patent Application Publication
(10) Pub. No.: US 2024/0379233 A1
(43) Pub. Date: Nov. 14, 2024

(54) Systems and Methods for Generating Reference Limits for Use in Diagnostic and Monitoring Systems

(71) Applicant: Cadwell Laboratories, Inc., Kennewick, WA (US)

(72) Inventors: Michal Holub, Block 707 (SG); Abram Gardner, San Clemente, CA (US); Stefan Stalberg, Helsingborg (SE)

(21) Appl. No.: 18/658,127

(22) Filed: May 8, 2024

Related U.S. Application Data
(60) Provisional application No. 63/500,884, filed on May 8, 2023.

(51) Int. Cl. G16H 50/20; A61B 5/00; A61B 5/388; A61B 5/397; G16H 10/60

(52) U.S. Cl. CPC G16H 50/20; A61B 5/388; A61B 5/397; A61B 5/7271; A61B 5/743; A61B 5/7435; A61B 5/748; G16H 10/60

(57) ABSTRACT

In systems and methods for dynamically generating reference limits for use in a neuromonitoring system, a first set of neuromonitoring data from separate patient tests conducted by the neuromonitoring system is received. Each of the separate patient tests is associated with variables. Each of the variables is associated with a corresponding one of the first neuromonitoring data as metadata. The first set of neuromonitoring data is compiled with associated metadata into one or more reference limits. When neuromonitoring data is received from a second patient test, at least one analysis is applied to the second patient test using the one or more reference limits.

### CROSS-REFERENCE

The present specification relies on U.S. Provisional Patent Application No. 63/500,884, titled “Systems and Methods for Generating Reference Limits for Use in Diagnostic/Monitoring Systems”, filed on May 8, 2023, for priority, the entirety of which is herein incorporated by reference.

### FIELD

The present specification is related generally to the field of neuromuscular diagnostic and monitoring systems. More specifically, the present specification is related to systems and methods for the dynamic generation of reference limits and novel corresponding data formats for use in neuromuscular diagnostic and monitoring systems.

### BACKGROUND

Several medical procedures involve using multiple sensors on the human body for the recording and monitoring of data required for patient care. Information, such as vital health parameters, cardiac activity, bio-chemical activity, electrical activity in the brain, gastric activity and physiological data, are usually recorded through on-body or implanted electrodes, or more generally sensors, which are controlled through a wired or wireless link. Typical patient monitoring systems comprise a control unit connected through a wire to one or more electrodes coupled to the specific body parts of the patient.

Neuromonitoring is the use of electrophysiological methods, such as electroencephalography (EEG), electromyography (EMG), and evoked potentials (EP), to monitor the function of certain neural structures (for example, nerves, spinal cord, brain and muscle). Neuromonitoring is used to evaluate abnormal brain electrical activity, and when applicable, to locate pathologic areas in the brain that can be resected or ablated. Some aspects of neuromonitoring involve placing EEG electrodes on a patient's scalp and within the patient's brain in order to record and monitor the electrical activity from various parts of the patient's brain. EEG procedures are classified as either non-invasive or invasive. In non-invasive EEG, a number of electrodes are deployed on a patient's scalp for recording electrical activity in portions of the underlying brain. In invasive EEG, through surgical intervention, the electrodes are placed directly in or on sections of the brain, in the form of a strip or grid, or are positioned in the deeper areas of the brain using depth electrodes. Each of these electrodes has multiple contacts wherein each of the multiple contacts is coupled to a wire lead which, in turn, is connected to a control unit adapted to receive and transmit electrical signals. The electrical activity captured by various electrodes is analyzed both visually and by using algorithms, in order to detect any abnormal brain activity and to localize the portion(s) of brain responsible for causing the specific activity or ailment.

A prospective reference limit study is the currently dominating methodology for determining reference limits in monitoring and electrodiagnostic testing. The analysis of retrospective data is still mostly used in a research context. On the other hand, in prospective studies, “normal” patients are selected to participate in clinical examinations to determine reference results. These reference results are then used to determine whether a patient's neuromonitoring results are indicative of a pathological condition or indicative of healthy physiology. Unfortunately, prospective data collection is time-consuming and costly, and most prospective studies have too few participants to stratify reference limits across multiple demographics, such as based on gender, ethnicity, body mass index (BMI) or age.

The generation of retrospective reference limits typically uses statistical analysis techniques to determine reference limits from standard clinical tests that include a mix of both healthy and unwell patients. This offers a much larger and robust data set without the use of additional time, that continuously grows with the number of tests available, and that allows for demographic stratification which includes parameters such as age, gender and BMI.

Currently, reference limits are created using prospective methods by testing healthy subjects, computing results and then manually entering the reference limits into diagnostic equipment. Some institutions have created reference limits retrospectively, but the data collection is typically a process where all clinical results are stored in a database, reference limits are manually computed, and subsequently manually entered into diagnostic software.

Accordingly, there is need for a quality-controlled, closed loop system that automates the process of collecting, analyzing, controlling, and inputting reference limits back into a diagnostic and monitoring system from which the data was collected. There is also a need to restructure data collection, storage, and analysis from being patient-centric to being test or procedure centric. Finally, there is a need to increase clinical quality, reduce cost, decrease clinician time, and minimize user errors.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a method of electrodiagnosis or neuromonitoring, comprising: selecting a electrodiagnostic or neuromonitoring device; applying at least one electrode to the patient, wherein the at least one electrode is in electrical communication with the electrodiagnostic or neuromonitoring device and wherein the at least one electrode is adapted to detect an electrical signal and transmit it to the electrodiagnostic or neuromonitoring device; acquiring data indicative of the patient's muscles and/or nerves based on the detected electrical signal; acquiring a reference limit, wherein the reference limit is generated by: receiving a selection of at least one of a plurality variables; acquiring electrodiagnostic or neuromonitoring data based on said selection of at least one of a plurality of variables, wherein the electrodiagnostic or neuromonitoring data was generated from a plurality of separate electrodiagnostic or neuromonitoring tests and wherein the electrodiagnostic or neuromonitoring data is associated with a plurality of variables; and applying one or more functions to the electrodiagnostic or neuromonitoring data to generate said reference limit; evaluating said data indicative of the patient's muscles and/or nerves based on the acquired reference signal; and causing a display of said evaluated data on a display.

Optionally, said plurality of variables comprise age, height, weight, gender, and condition.

Optionally, at least a portion of the plurality of variables is stored as metadata in relation to the electrodiagnostic or neuromonitoring data. Optionally, the display is further configured to display an option to select one or more portions of the electrodiagnostic or neuromonitoring data and to display said selected one or more portions of the electrodiagnostic or neuromonitoring data with associated metadata.

Optionally, said acquisition of the reference limit is further generated by displaying the electrodiagnostic or neuromonitoring data to a user prior to applying the one or more functions. Optionally, the method further comprises receiving from the user a selection of a portion of the electrodiagnostic or neuromonitoring data.

Optionally, the method further comprises including said data indicative of the patient's muscles and/or nerves in the electrodiagnostic or neuromonitoring data.

Optionally, the one or more functions comprises extracting a portion of the electrodiagnostic or neuromonitoring data and generating a histogram. Optionally, the one or more functions further comprises extracting a portion of the electrodiagnostic or neuromonitoring data and applying a normalization process to minimize a skewness of a distribution of the extracted portion of the electrodiagnostic or neuromonitoring data. Optionally, the one or more functions further comprises, after applying said normalization process, applying a regression analysis on said normalized extracted portion of the electrodiagnostic or neuromonitoring data. Optionally, the one or more functions further comprises, after applying said regression analysis, determining Z scores on said normalized extracted portion of the electrodiagnostic or neuromonitoring data. Optionally, the one or more functions further comprises applying a smoothing function to said Z scores. Optionally, the one or more functions further comprises generating a cumulative distribution function plot using said smoothed Z scores. Optionally, the one or more functions further comprises determining a slope of a portion of the cumulative distribution function plot. Optionally, the one or more functions further comprises determining a normal zone of the cumulative distribution function plot and identifying at least one inflection point where the cumulative distribution function plot enters the normal zone. Optionally, the one or more functions further comprises determining a slope of a portion of the cumulative distribution function plot.

Optionally, the one or more functions comprises extracting a portion of the electrodiagnostic or neuromonitoring data and generating a plot, wherein said plot comprises Z scores derived from the electrodiagnostic or neuromonitoring data. Optionally, the one or more functions comprises determining one or more inflection points in said plot, wherein said one or more inflection points are indicative of boundaries between an abnormal measurement range and a normal measurement range.

Optionally the method further comprises generating a graphical user interface, wherein said graphical user interface is configured to display a plurality of reference limits and wherein each of said plurality of reference limits is displayed using a set of parameters.

Optionally, the set of parameters comprises two or more of degree of quality, age groups, types of test, anatomies, measured test parameters, normal ranges of values for the measured test parameter, and model. Optionally, the degree of quality is color coded, and the measured test parameter comprises conduction velocity.

In some embodiments, the present specification is directed towards a non-transient computer readable medium configured to store a plurality of programmatic instructions, wherein, when executed, the programmatic instructions cause a computing device to: receive a first set of neuromonitoring data from a plurality of separate patient tests, wherein each of the plurality of separate patient tests is associated with a plurality of variables; associate each of the plurality of variables with a corresponding one of the first neuromonitoring data as metadata; compile said first set of neuromonitoring data with associated metadata into one or more reference limits; receive neuromonitoring data from a second patient test; and apply at least one analysis to the second patient test using the one or more reference limits.

Optionally, the neuromonitoring data is electromyography data.

Optionally, the plurality of variables comprises a patient age, a patient height, and a patient gender.

Optionally, the plurality of variables further comprises at least one of a type of test, a patient weight, or a patient condition.

Optionally, the non-transient computer readable medium further comprises programmatic instructions that, when executed, generate a display and wherein said display is configured to present an option to select one or more portions of the first set of neuromonitoring data with associated metadata.

Optionally, the non-transient computer readable medium further comprises programmatic instructions that, when executed, compile the selected one or more portions of the first set of neuromonitoring data with associated metadata into one or more reference limits.

Optionally, the first set of neuromonitoring data includes clinical data corresponding to normal and abnormal test results.

In some embodiments, the present specification is directed towards a method of dynamically generating reference limits for use in a neuromonitoring system, comprising: receiving a first set of neuromonitoring data from a plurality of separate patient tests conducted by the neuromonitoring system, wherein each of the plurality of separate patient tests is associated with a plurality of variables; associating each of the plurality of variables with a corresponding one of the first neuromonitoring data as metadata; compiling said first set of neuromonitoring data with associated metadata into one or more reference limits; receiving neuromonitoring data from a second patient test; and applying at least one analysis to the second patient test using the one or more reference limits.

Optionally, the neuromonitoring data is electromyography data.

Optionally, the plurality of variables comprises a patient age, a patient height, and a patient gender.

Optionally, the plurality of variables further comprises at least one of a type of test, a patient weight, or a patient condition.

Optionally, the method further comprises generating a display, wherein said display is configured to present an option to select one or more portions of the first set of neuromonitoring data with associated metadata.

Optionally, the method further comprises compiling the selected one or more portions of the first set of neuromonitoring data with associated metadata into one or more reference limits.

In some embodiments, the present specification is directed toward a system for dynamically generating reference limits, comprising: a neuromonitoring system configured to generate a first set of neuromonitoring data from a plurality of separate patient tests; a database system in data communication with the neuromonitoring system, wherein the first set of neuromonitoring data is saved in the database system; a computing device in data communication with the neuromonitoring system and the database system, wherein the computing device includes a non-transient computer readable medium configured to store a plurality of programmatic instructions, which when executed cause the computing device to: receive the first set of neuromonitoring data from the database system, wherein each of the plurality of separate patient tests is associated with a plurality of variables; associate each of the plurality of variables with a corresponding one of the first neuromonitoring data as metadata; compile said first set of neuromonitoring data with associated metadata into one or more reference limits, wherein the one or more reference limits are exported into the neuromonitoring system; receive neuromonitoring data from a second patient test; and apply at least one analysis to the second patient test using the one or more reference limits.

Optionally, the neuromonitoring data is electromyography data.

Optionally, the plurality of variables comprises a patient age, a patient height, and a patient gender. Optionally, the plurality of variables further comprises at least one of a type of test, a patient weight, or a patient condition.

Optionally, the computing device further comprises programmatic instructions that, when executed, generate a display wherein said display is configured to present an option to select one or more portions of the first set of neuromonitoring data with associated metadata. Optionally, the computing device further comprises programmatic instructions that, when executed, compile the selected one or more portions of the first set of neuromonitoring data with associated metadata into one or more reference limits.

Optionally, the first set of neuromonitoring data includes clinical data corresponding to normal and abnormal test results.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a block diagram illustration of a closed loop RLG (Reference Limits Generator) system, in accordance with some embodiments of the present specification;

FIG. 1B is a workflow implemented in the RLG system, in accordance with some embodiments of the present specification;

FIG. 1C is a flowchart detailing a plurality of steps of a method for dynamically generating reference limits for use in an electrodiagnostic or neuromonitoring system, in accordance with some embodiments of the present specification;

FIG. 1D is a flowchart describing a plurality of steps of a method for compiling one or more reference limits, in accordance with some embodiments of the present specification;

FIG. 2A shows a plot of a mixed clinical dataset distribution, in accordance with some embodiments of the present specification;

FIG. 2B is a graphical representation of the application of normalization methods to various data distributions, in accordance with some embodiments of the present specification;

FIG. 3A shows a plurality of plots related to a multiple regression model, in accordance with some embodiments of the present specification;

FIG. 3B is a combined graph showing that the normal mean varies depending on the combination of selected predictors, in accordance with some embodiments of the present specification;

FIG. 4A shows a first plot related to a transformation from measured test parameter values to Z-scores, in accordance with some embodiments of the present specification;

FIG. 4B shows a second plot related to a transformation from measured test parameter values to Z-scores, in accordance with some embodiments of the present specification;

FIG. 4C shows a third plot related to a transformation from measured test parameter values to Z-scores, in accordance with some embodiments of the present specification;

FIG. 5 shows a plurality of steps in generating a normal reference limits model, in accordance with some embodiments of the present specification;

FIG. 6A is a first view of a GUI configured to enable a clinician to quality check and validate generated reference limits, in accordance with some embodiments of the present specification;

FIG. 6B is a second view of the GUI of FIG. 6A, in accordance with some embodiments of the present specification;

FIG. 6C shows the validated reference limits, from the GUI of FIG. 6A, being exported to a diagnostic/monitoring system, in accordance with some embodiments of the present specification;

FIG. 6D shows a plurality of histogram data being displayed in an area or portion of the GUI of FIG. 6A, in accordance with some embodiments of the present specification;

FIG. 6E shows a plurality of comparative graphs being displayed in the area or portion of the GUI of FIG. 6A, in accordance with some embodiments of the present specification; and

FIG. 7 shows another GUI generated by the RLG module to enable a user, such as a clinician, to configure a database synchronizer, in accordance with some embodiments of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

The term “module”, “application”, “component” or “engine” used in this disclosure may refer to computer logic utilized to provide a desired functionality, service or operation by programming or controlling a general purpose processor. Stated differently, in some embodiments, a module, application or engine implements a plurality of instructions or programmatic code to cause a general purpose processor to perform one or more functions. In various embodiments, a module, application or engine can be implemented in hardware, firmware, software or any combination thereof. The module, application or engine may be interchangeably used with unit, logic, logical block, component, or circuit, for example. The module, application or engine may be the minimum unit, or part thereof, which performs one or more particular functions.

The term “reference limits” used in this disclosure refers to a set of values providing a benchmark against which another patient's test results are compared to determine if they are within, or outside, of an expected value or value range for that patient's demographics (sex, age), condition, anatomical location, type of test, among other factors.

The term “clinical results” or “patient clinical results data” used in this disclosure refers to values generated from different clinical examinations that quantify and/or describe physiological characteristics of a patient.

The term “demographics” used in this disclosure refers to data used to categorize individuals for identification, clinical use, records matching, and other purposes for instance name, age, height, geographic location, weight, ethnicity, body mass index (BMI), gender, and other descriptors.

The term “statistical analysis” used in this disclosure refers to collecting, analyzing, and/or presenting large amounts of data to discover underlying patterns and trends.

The term “diagnostic and/or monitoring system” used in this disclosure refers to a system consisting of software or combined software and hardware dedicated to creating clinical test results. The system includes electrophysiological methods, such as electroencephalography (EEG), electromyography (EMG), and evoked potentials (EP), to monitor the function of certain neural structures (for example, nerves, spinal cord, brain and muscle). For instance, electromyography equipment is configured to record and analyze physiological signals from the body and generate different types of test results such as, but not limited to, nerve conduction velocity and muscle response latencies.

The term “results database” used in this disclosure refers to a computing device configured as a database to collect, organize, and store measured clinical data from the performed examinations.

The term “electromyography data” used in this disclosure refers to the measurement of myoelectric activity, or muscle electrical signals, in units of microvolts.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

It should be noted herein that the functionalities described herein apply to at least diagnostic and monitoring products that generate quantitative results that are anatomically uniquely identifiable. In embodiments, test types include, but are not limited to nerve conduction studies (NCS+) and neuromuscular ultrasound studies (NMUS).

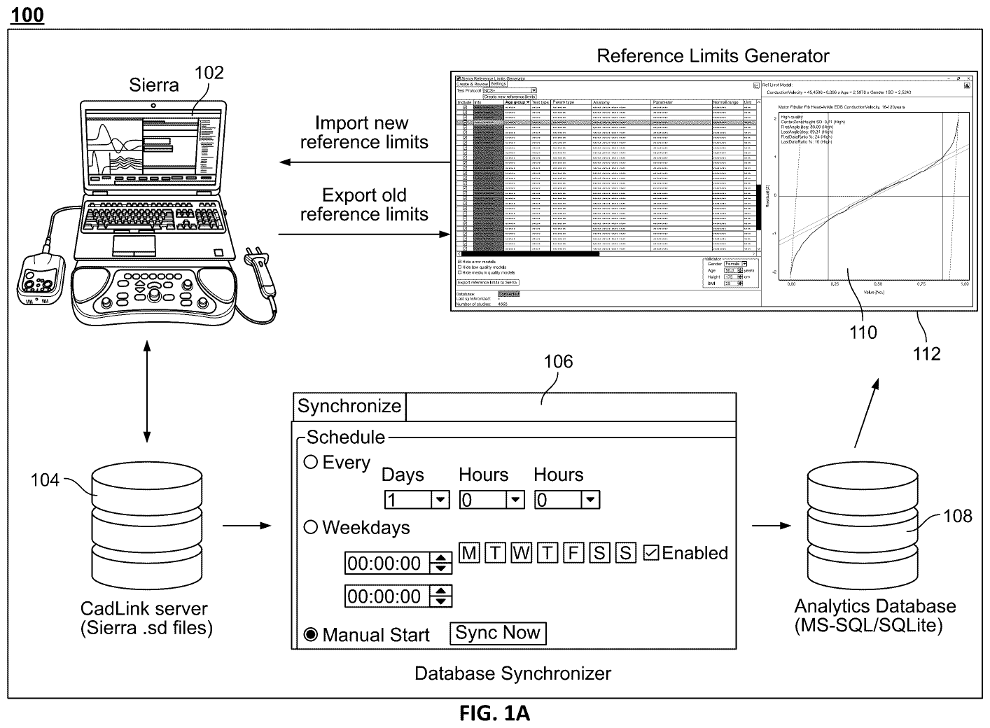

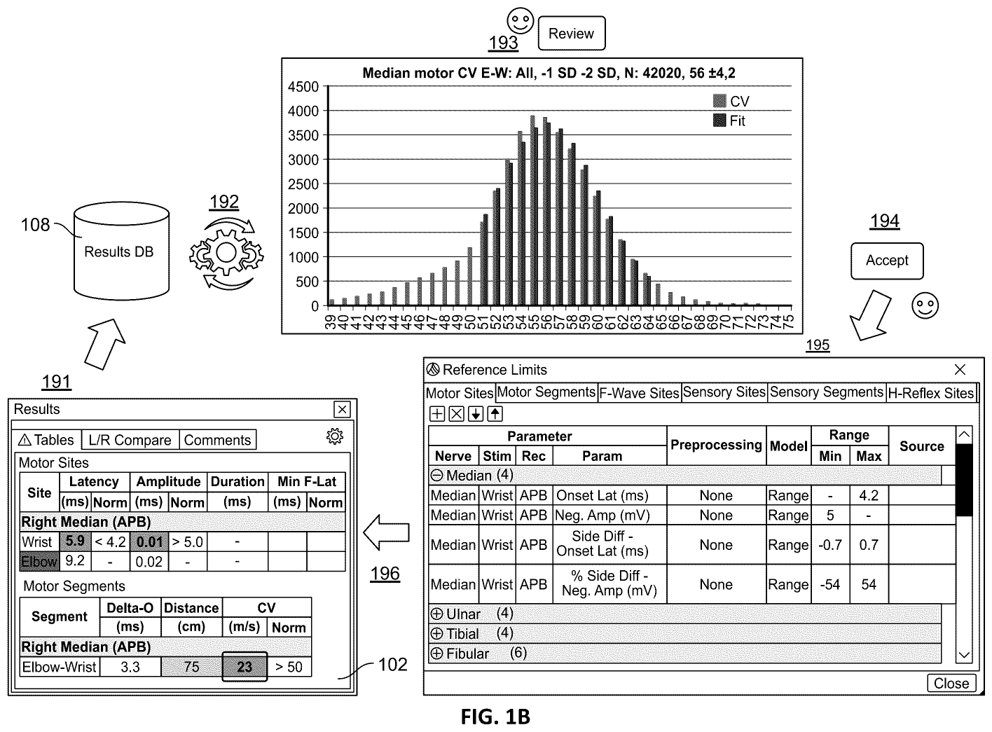

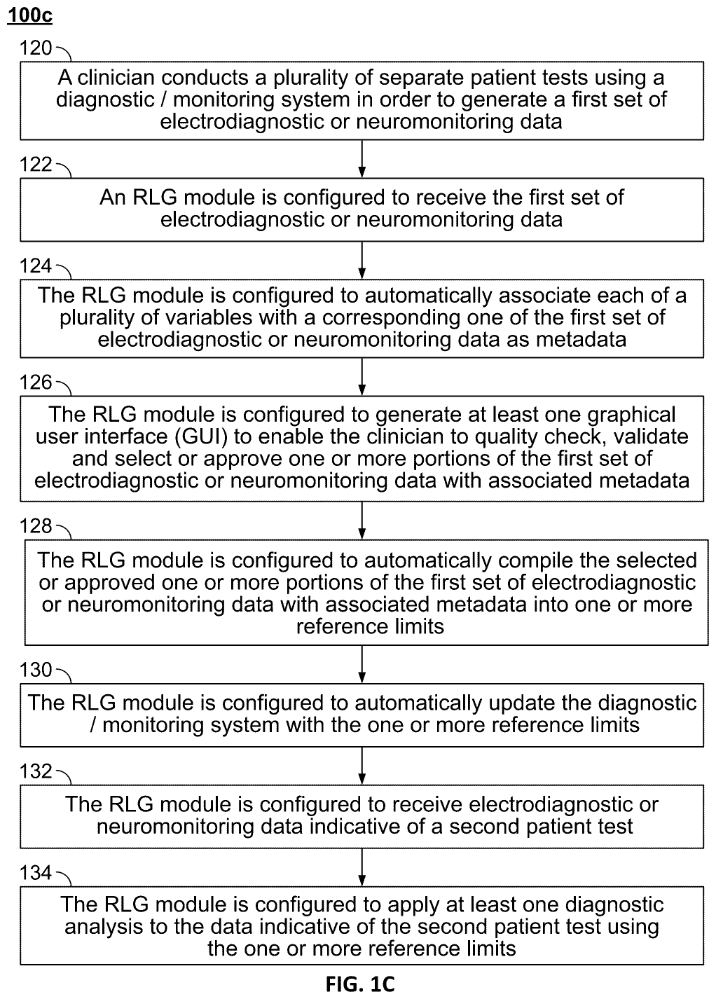

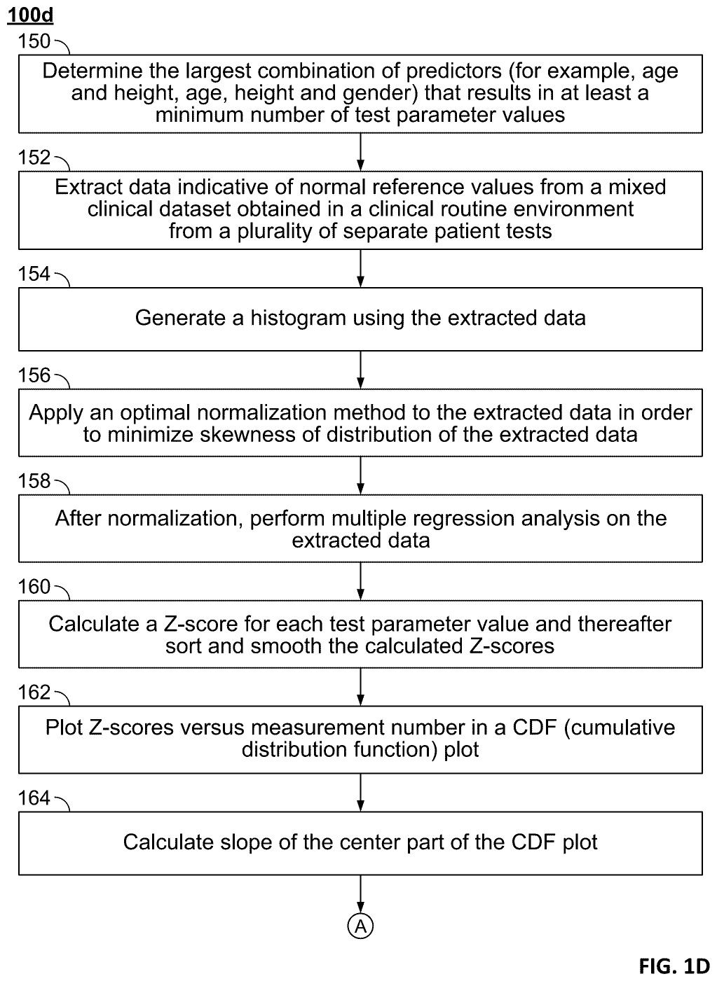

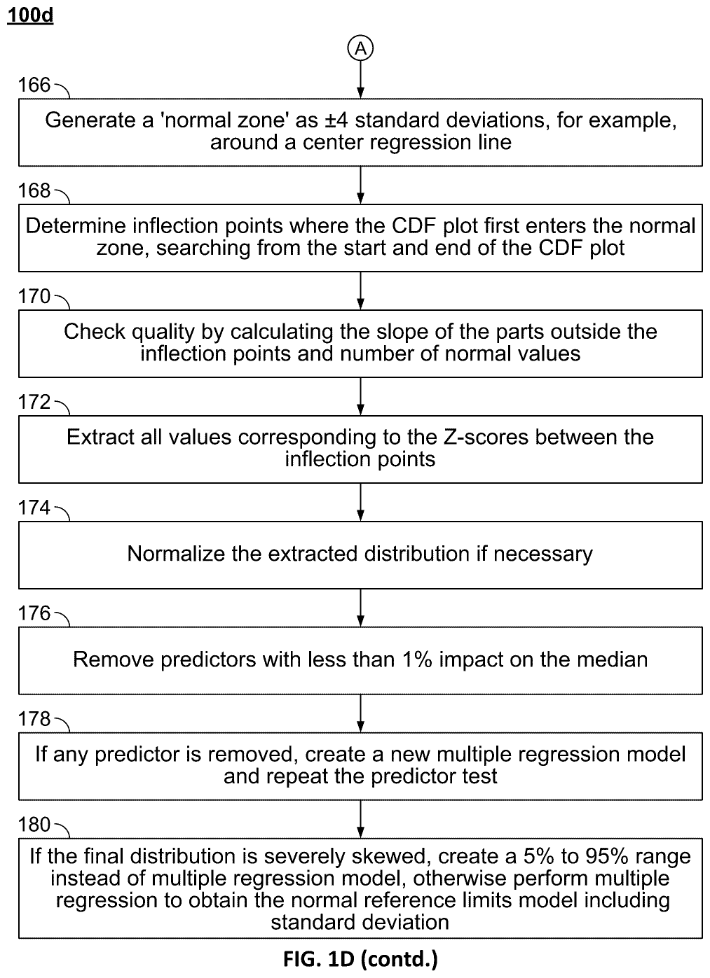

FIG. 1A is a block diagram illustration of a closed loop RLG (Reference Limits Generator) system 100, in accordance with some embodiments of the present specification. The system 100 comprises a diagnostic and/or monitoring system 102, such as an electrodiagnostic or neuromonitoring system, in data communication with at least one server 104. Patient clinical results data (also referred to hereinafter as “results”) generated by the diagnostic and/or monitoring system 102 is either saved locally in the diagnostic and/or monitoring system 102 or on the at least one server 104. A database synchronizer 106 is configured to receive and read patient clinical results data and save all results in a results database system 108. Current reference limits in the diagnostic and/or monitoring system 102 are optionally exported to an RLG module, application or engine 110, implemented on a computing device 112 in data communication with the diagnostic and/or monitoring system 102. In some embodiments, new reference limits are created by the RLG module 110 based on data in the results database system 108. The new reference limits are imported to the diagnostic and/or monitoring system 102 for immediate use.

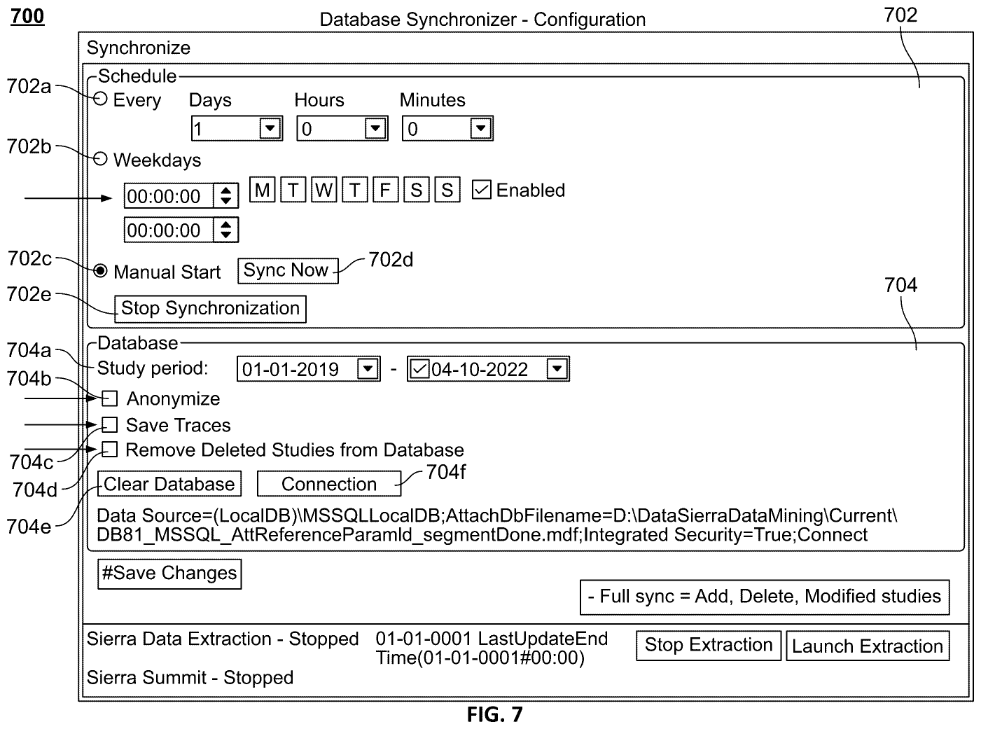

FIG. 7 shows a GUI 700 generated by the RLG module 110 to enable a user/clinician to configure the database synchronizer 106 (FIG. 1A), in accordance with some embodiments of the present specification. As shown, a first area or portion 702 is configured to enable the clinician to choose one of three options that define a schedule at which the database synchronizer should read patient clinical results data and save all results in database system 108. A first option 702a allows the clinician to schedule automatic data synchronization in days, hours, and/or minutes. A second option 702b allows the clinician to schedule automatic data synchronization in terms of weekdays. A third option 702c allows the clinician to schedule data synchronization manually on actuating a graphical visual element 702d. Data synchronization may be terminated, at any point in time, by actuating a graphical visual element 702e.

A second area or portion 704 is configured to provide at least one of the following: a fourth option 704a to select a study period (such as a date range) for patient clinical results data, a fifth option 704b which, when enabled, is configured to cause anonymization of patient clinical results data, a sixth option 704c which when enabled, is configured to cause traces to be saved, and a seventh option 704d which, when enabled, is configured to cause deleted studies to be removed from the database system 108. It should be noted that the traces to be saved include raw trace data indicative of brain electrical activity in different regions, in addition to the numerical results to support research or analysis. A visual graphical element 704e is configured to cause, when selected, previous selections of all options in the second area or portion 704 to be cleared. A visual graphical element 704f is configured to cause the clinical database system 108 to be connected to the diagnostic and/or monitoring system 102 when selected, which supports the syncing of the clinical database with the analytics database.

In embodiments, the RLG module 110 is configured to implement a plurality of programmatic instructions or code for restructuring how data is packaged and stored, transitioning away from a patient centric model where the results, demographics, test type, condition, and other variables are structured to describe a patient to a results-centric model where the demographics, test type, condition, and other variables function as metadata in relation to the results, as will be further described herein. Further to this, a clinical workflow and functionality afforded by the RLG module 110 includes, but is not limited to, complete integration, automatic quality indication, automatic comparison with existing reference data, and if approved by the user, automatic update of clinical equipment with new or updated reference limits.

Thus, in accordance with aspects of the present specification, the RLG module 110 is configured to automatically derive reference limits from routine clinical examinations using the diagnostic and/or monitoring system 102, display the reference limits to a clinician for review and approval, and update the diagnostic and/or monitoring system 102 with the approved reference limits in the closed loop system 100 with the following process and features:
a) Patient clinical results data are collected and stored in the clinical results database system 108 with relevant anatomical, physiological, and diagnostic identification and labelling.
b) The collected patient clinical results data is automatically analyzed by the RLG module 110 to derive retrospective reference limits stratified by patient demographics such as weight, BMI (Body Mass Index), height, gender and age.
c) Upon clinician approval, the new or updated reference limits are automatically inserted into the diagnostic and/or monitoring system 102 and used in future diagnostic examinations.

In various embodiments, the RLG module 110 is configured to create normal reference limits from mixed material clinical data, containing both normal and abnormal results, recorded with the diagnostic and/or monitoring system 102. For example, a patient's results may be indicative of abnormal functioning with respect to both hands, yet normal functioning of both legs—therefore the normal data related to the legs may be automatically extracted and used for deriving reference limits. Reference limits for all studies, anatomies and selected parameters are generated automatically. Graphs and numerical results are presented to the user for validation purposes. Accepted or approved reference limits are imported into the diagnostic and/or monitoring system 102 for immediate use.

It should be appreciated that diagnostic and/or monitoring system electronically accesses reference limit data which may be stored in a memory local to the diagnostic and/or monitoring system or remotely therefrom. The presently disclosed invention automatically restructures collected data, as described herein, and updates the reference limit data stored in the local or remote memory. Accordingly, the RLG system 100 is configured such that it enables a simple and efficient way for clinicians to create reliable clinical reference limits that are based on their specific examination techniques, equipment, and patient population.

The RLG system 100 is advantageous for deriving reference limits for a host of reasons, including, but not limited to the following advantages: 1) the number of recordings can be large enough to accurately stratify reference values by age, gender, weight, BMI, height, and other demographic characteristics; 2) local variations in diagnostic and/or monitoring techniques can be accommodated; 3) local variations in patient demographics can be accommodated; 4) reference values can be made available for different patient categories that are very difficult to maintain in prospective studies (infants and young children as well as older population); 5) reference values can be continuously improved as number of examinations increase; 6) the collection, analysis and input of the patient clinical results data are done with minimal user input; 7) a user can choose to see updated reference values at any time; 8) after a user accepts the reference limits they may be automatically applied to the corresponding test protocols, eliminating manual data input and reducing errors; and 9) the generated reference limits can be used for quality control by statistical comparison of test result variation between different technicians, physicians, and/or facilities.

FIG. 1B is a diagram of a workflow implemented in the closed loop RLG system 100, in accordance with some embodiments of the present specification. At step 191, clinicians perform a patient's examination, using the diagnostic and/or monitoring system 102, and the data representing the results are automatically saved to the results clinical database system 108. The results data are labeled with anatomical, physiological, and diagnostic relevant identification. At step 192, data contained in the results database system 108 are analyzed by the RLG module 110 either continuously or at user defined time and periodicity to generate reference limits stratified by various patient demographics. The analysis can be manually or automatically updated as more patient clinical results data is collected in the results database system 108. At step 193, the generated reference limits are managed in a plurality of ways such as, for example, a) a user can manually at any point in time decide to review the currently available reference limits, b) the RLG module 110 can automatically inform the user when new reference limits are available, and c) the RLG module 110 can automatically notify the user when new reference limits are available that deviate a predefined amount from the currently used reference limits.

At step 194, the RLG module 110 enables the user to evaluate the quality of the new reference values in a plurality of ways such as, for example: a) the RLG module 110 can provide different quality measures qualifying the suggested reference limits, b) the RLG module 110 can show a comparison between currently used and the suggested reference values, and c) the RLG module 110 presents graphical and numerical reference values for a user definable sample patient.

At step 195, clinicians indicate which of the suggested reference limits they approve based on their review of the data presented. At step 196, the approved reference limits are automatically imported into the diagnostic/monitoring System 102 and used when new examinations are performed.

FIG. 1C is a flowchart describing a plurality of steps of a method 100c for dynamically generating reference limits for use in an electrodiagnostic or neuromonitoring system, in accordance with some embodiments of the present specification. In embodiments, the method 100c is implemented in the closed loop RLG system 100 of FIG. 1A with reference to the workflow of FIG. 1B. At step 120, a clinician conducts a plurality of separate patient tests using the diagnostic and/or monitoring system 102 to generate a first set of electrodiagnostic or neuromonitoring data. In embodiments, each of the plurality of separate patient tests include, but are not limited to, at least one of a nerve conduction test, EMG test, evoked potentials test, neuromuscular ultrasound test, single fiber electromyography (SFEMG) test, repetitive nerve stimulation test, blink response test, and any assessment of muscle fiber or nerve action potentials. In embodiments, the test parameters for each of the plurality of separate patient tests include, but are not limited to, at least one of latency, amplitude, area, durations, frequency, echo intensity, angle, volume and other electrical parameters. In various embodiments, each of the plurality of separate patient tests is associated with a plurality of variables such as, but not limited to, at least one of a patient's age, height, weight, gender, BMI, ethnicity, geographic location, condition, class of test (such as, for example, motor, sensory), anatomies (such as, for example, recorded nerves, stimulation, and recording positions) or other demographics. Stated differently, the plurality of variables may comprise demographic, anatomical, physiological, and/or diagnostic relevant identification.

At step 122, the RLG module 110 is configured to receive the first set of electrodiagnostic or neuromonitoring data. At step 124, the RLG module 110 is configured to automatically associate each of the plurality of variables with a corresponding one of the first set of electrodiagnostic or neuromonitoring data as metadata. This restructuring of data runs counter to conventional approaches. Typically, test data is stored in the form of a patient record where the data file comprises a patient identifier, all associated descriptors of the patient, and the test information. If using a relational database, the patient identifier therefore would be the primary key, or primary keyword, that distinctively identifies the record. For a given record, a relational database has only one primary key around which the record is built. The presently disclosed system, however, creates a file or data record built around the type of test where the demographic data, such as the patient's age, height, weight, gender, BMI, ethnicity, geographic location, condition, or anatomies (such as, for example, recorded nerves, stimulation, and recording positions), are the metadata. If using a relational database, the type of test therefore would be the primary key, or primary keyword, that distinctively identifies the record. The restructuring of data around the type of test allows for the real-time selection of variables (i.e. gender and age), real-time selection of test records based on the selected variables, and the real-time generation of appropriate reference limits, and use thereof, based on those selections. In one embodiment, the presently disclosed electrodiagnosis or neuromonitoring process concurrently or serially generates both a patient record and a separate test or study record with demographic data associated thereto.

At step 126, the RLG module 110 is configured to generate at least one graphical user interface (GUI) that allows the clinician to quality check, validate, and select or approve one or more portions of the first set of electrodiagnostic or neuromonitoring data with associated metadata.

At step 128, the RLG module 110 is configured to automatically compile the selected or approved one or more portions of the first set of electrodiagnostic or neuromonitoring data with associated metadata into one or more reference limits. In various embodiments, the RLG module 110 is configured to apply a plurality of statistical methods and principles in order to compile the one or more reference limits. In embodiments, the plurality of statistical methods and principles may include multiple regression analysis, Z-score plots, mixed distribution analysis, normalized data sets, or any other principles as are known to those of ordinary skill in the art.

At step 130, the RLG module 110 is configured such that it automatically updates the diagnostic/monitoring system 102 with the one or more reference limits.

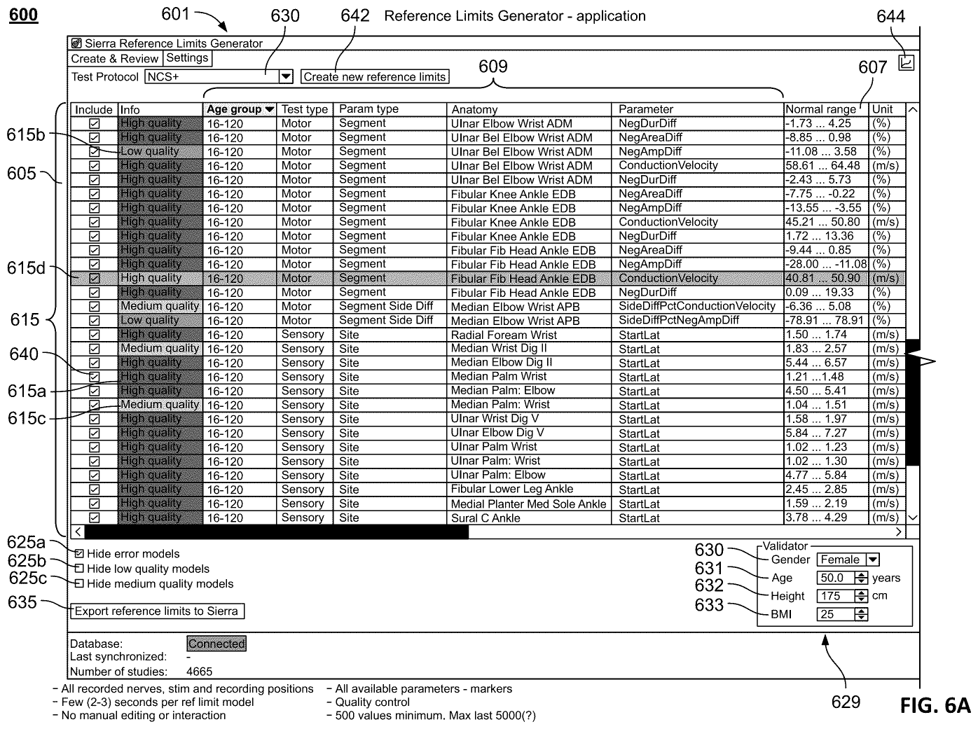

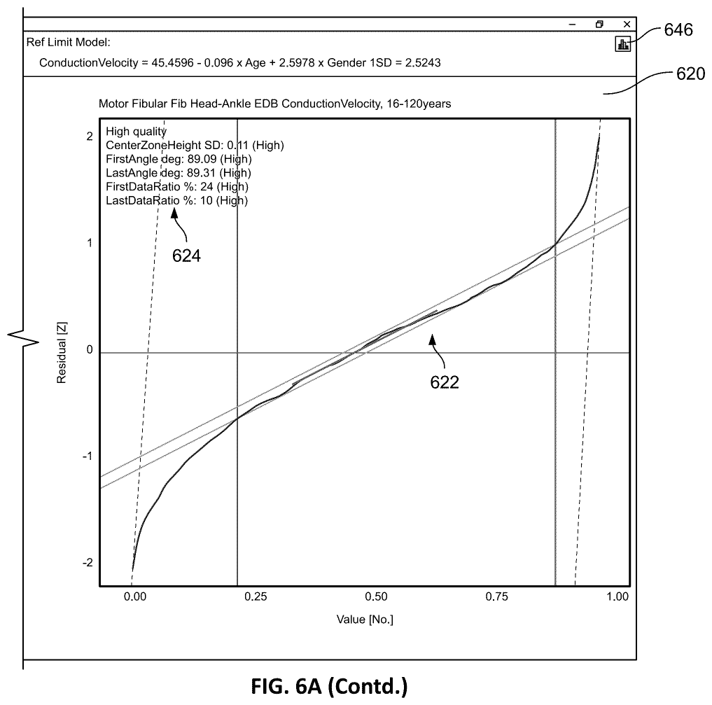

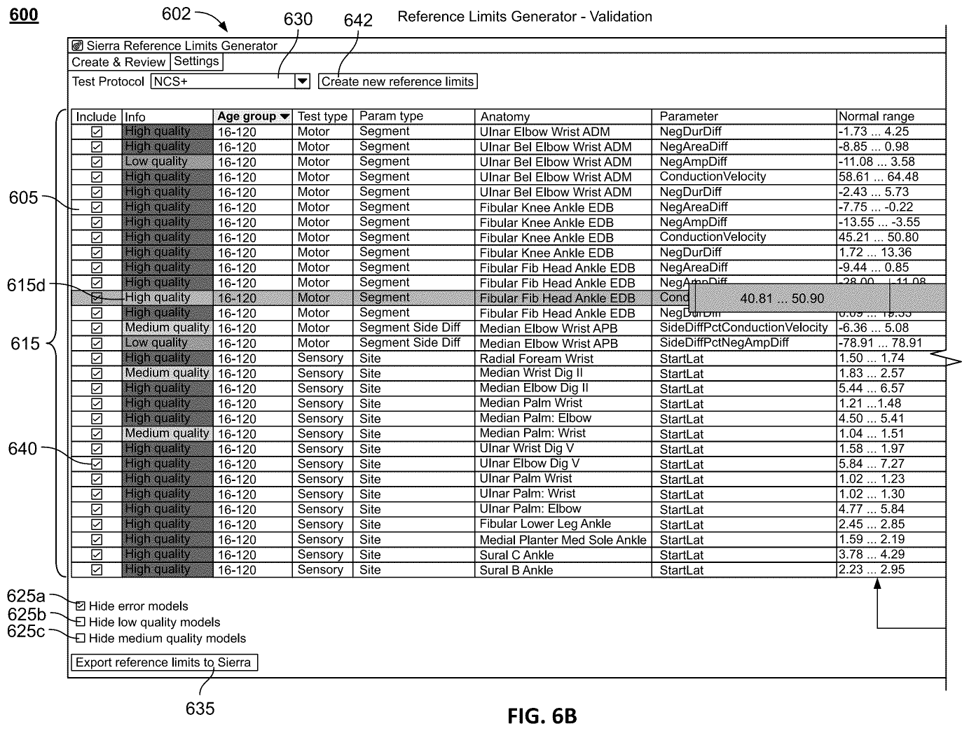

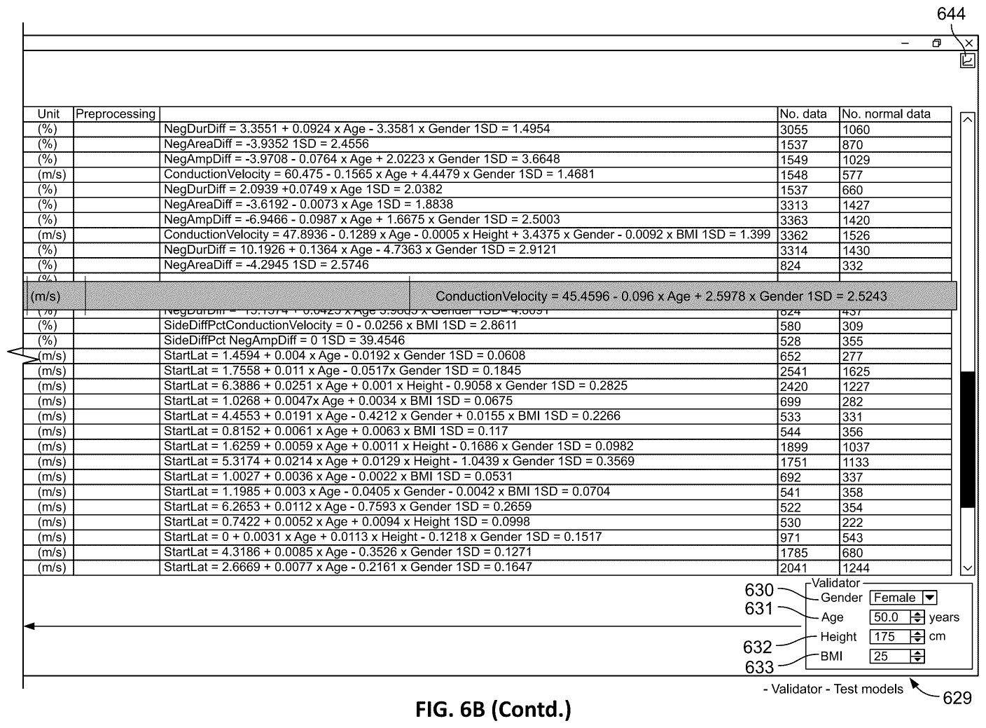

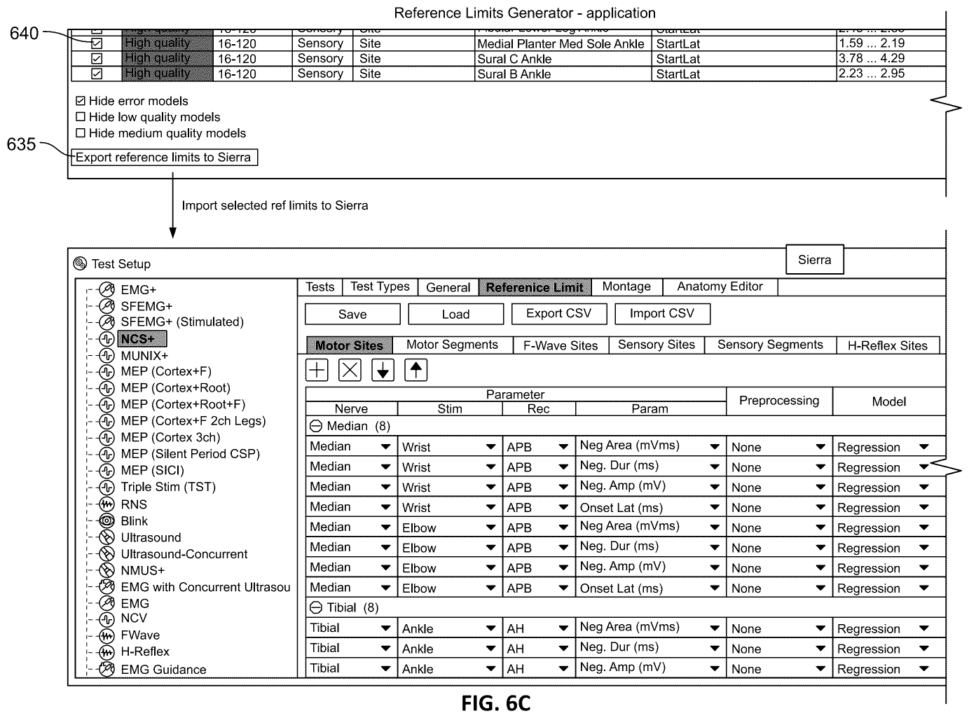

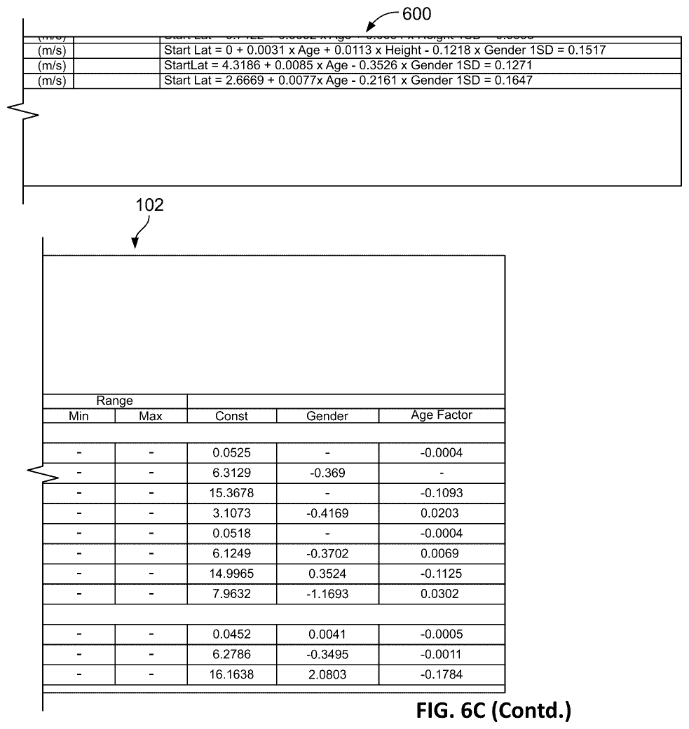

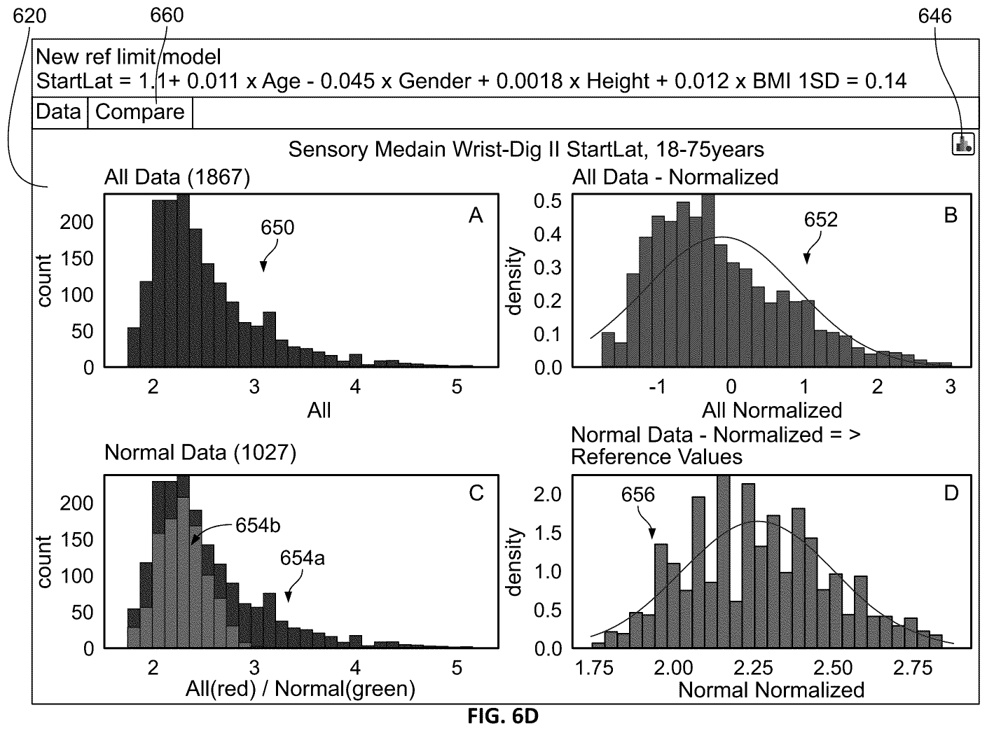

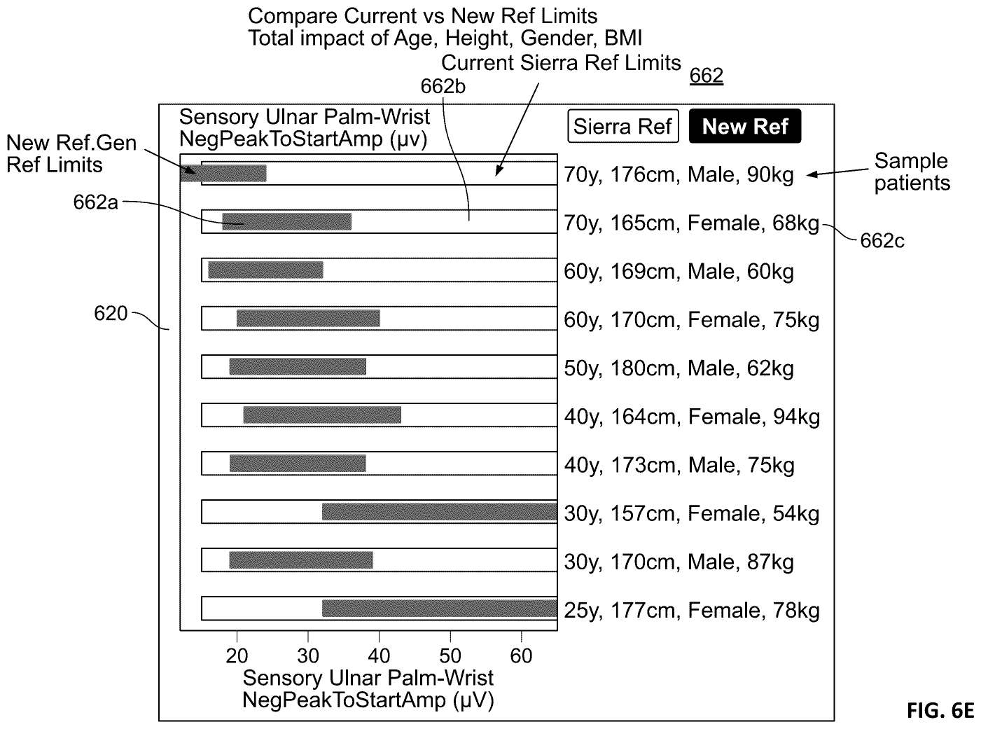

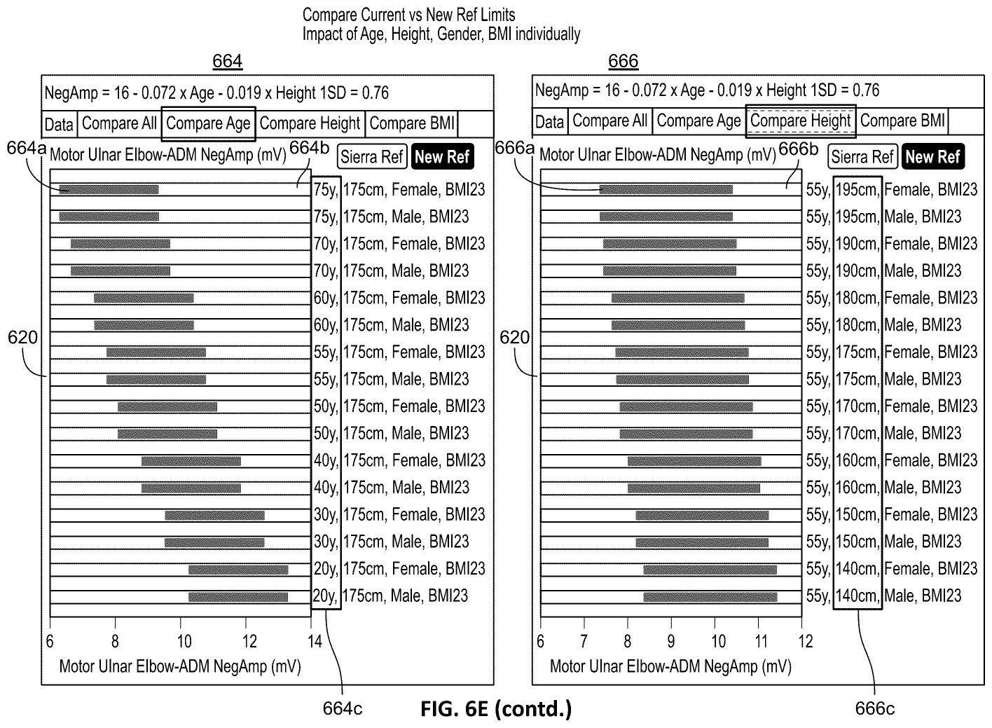

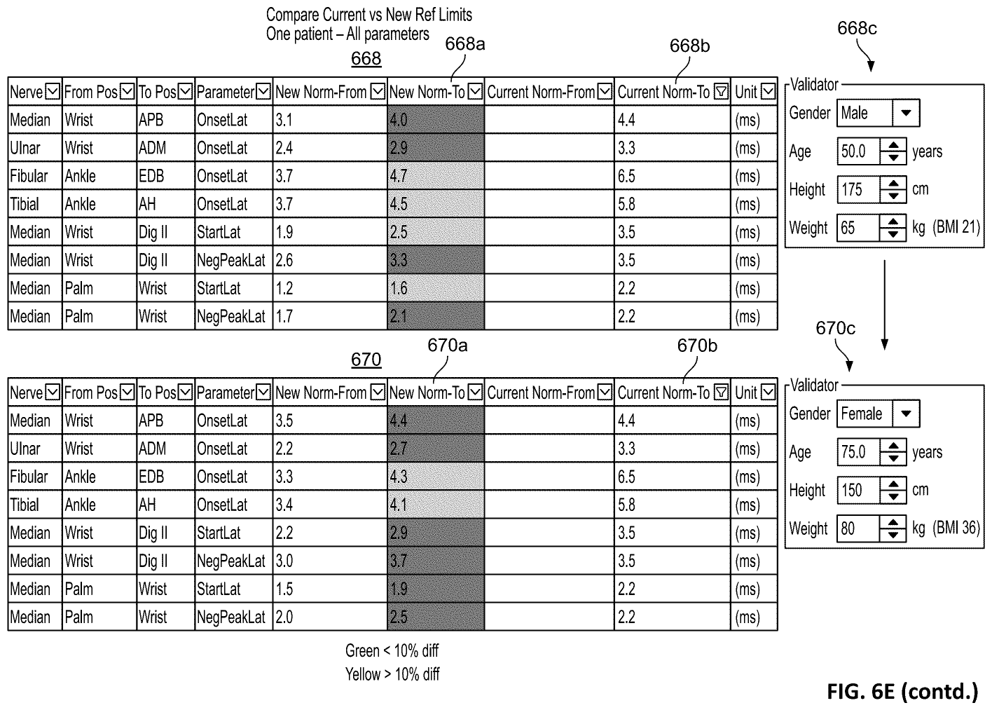

FIG. 6A is a first view 601 and FIG. 6B is a second view 602 of a GUI 600 configured to enable a clinician to quality check and validate generated reference limits, in accordance with some embodiments of the present specification. Referring now to FIGS. 6A and 6B, as shown, the first view 601 includes a first area or portion 605 and a drop down list 630, positioned above the first area or portion 605, that can be manipulated to select a test protocol from a list of protocols (such as, for example, nerve conduction studies) for which reference limits need to be generated. A total number of studies, corresponding to the selected test protocol, are initially displayed in the first area or portion 605. Thereafter, clicking a visual graphical element 642 (titled ‘create new reference limits’) causes the RLG module 110 to automatically generate reference limits for all anatomies and parameters. Consequently, the first area or portion 605 displays a plurality of data records 615 where each of the plurality of data records 615 includes a reference limit 607 along with a plurality of associated metadata 609.

In some embodiments, each of the plurality of data records 615 is coded in order to indicate a level of quality. In embodiments, a color coding is used. For example, a data record 615a may be coded with a first color (such as green) indicative of a ‘high quality’ since the amount of underlying electrodiagnostic or neuromonitoring data is enough or sufficient (for example, the amount of underlying electrodiagnostic or neuromonitoring data is at least sufficient for determining a reliable reference limit), a data record 615b may be coded with a second color (such as red) indicative of a ‘low quality’ since the amount of underlying electrodiagnostic or neuromonitoring data is not enough (for example, the amount of underlying electrodiagnostic or neuromonitoring data is not sufficient for determining reliable reference limits), and a data record 615c may be coded with a third color (such as yellow) indicative of a ‘medium quality’ since the amount of underlying electrodiagnostic or neuromonitoring data is short of being sufficient but better than not being enough (for example, the amount of underlying electrodiagnostic or neuromonitoring data lies in a range that would present reliable reference limits.

In embodiments, any of the plurality of data records 615 may be selected and thereafter a visual graphical element 644, positioned above the first area or portion 605, which, when actuated or enabled is configured to cause a display of a second area or portion 620. The second area or portion 620 then, by default, is used to display a plurality of Z-scores sorted by value and plotted versus measurement number to generate a CDF (cumulative distribution function) plot 622 corresponding to a selected data record 615d (and, therefore, for a specific parameter and anatomy). An associated plurality of numerical data 624 is also displayed in association with the CDF plot 622 (graphical data). To hide the second area or portion 620, as shown in the second view 602, the visual graphical element 644 is disabled.

A plurality of histogram data, corresponding to the selected data record 615d, may additionally be displayed in the second area or portion 620 upon actuating or enabling a visual graphical element 646 positioned in the second area or portion 620. As shown in FIG. 6D, the plurality of histogram data displayed includes: a first histogram data 650 of all mixed material clinical data, a second histogram data 652, after normalization, of all mixed material clinical data, a third histogram data of all mixed material clinical data (shown in a first color 654a) overlaid with extracted normal values (shown in a second color 654b), and a fourth histogram data 656 of the normal values after normalization. To hide the plurality of histogram data the visual graphical element 646 is disabled.

In some embodiments, the RLG module 110 is configured to generate a plurality of comparative graphs to allow a user to compare current reference limits with the new reference limits created by the RLG module 110. For this, a visual graphical element 660 (shown in FIG. 6D), which may be titled ‘compare’ or have a similar title, which, when actuated or enabled is configured to generate the plurality of comparative graphs in the second area or portion 620. Consequently, as shown in FIG. 6E, a first comparative graph 662 shows new reference limits 662a against current reference limits 662b for a sample group of patients with different demographics 662c (age, height, gender and BMI). A second comparative graph 664 shows new reference limits 664a against current reference limits 664b for a sample group of patients on the basis of a first single demographic 664c such as, age. A third comparative graph 666 shows new reference limits 666a against current reference limits 666b for a sample group of patients on the basis of a second single demographic 666c such as, height. Thus, separate comparative graphs, for a sample group of patients, may be generated and displayed on the basis of each of the demographics, such as age, height, gender and BMI. A fourth comparative graph 668 shows new reference limits 668a against current reference limits 668b for a single sample patient on the basis of a first set of values for various demographics 668c. Similarly, a fifth comparative graph 670 shows new reference limits 670a against current reference limits 670b for another single sample patient on the basis of a second set of values for various demographics 670c.

Referring back to FIGS. 6A and 6B, in some embodiments, the clinician may hide or deselect those of the plurality of data records 615 that are indicative of error models, low quality models and/or medium quality models. This is accomplished by the clinician selecting one or more of a first check-box 625a indicative of excluding or hiding data records related to error models, a second check-box 625b indicative of excluding or hiding data records related to low quality models and a third check-box 625c indicative of excluding or hiding data records related to medium quality models.

To test a multiple regression model, a patient with specific demographics must be specified. The test patient is specified with a plurality of validator fields 629, positioned below the first area or portion 605, that can be manipulated to choose gender, age, height and BMI in order to display reference limits in the corresponding plurality of data records 615 characterized by the chosen gender, age, height and BMI. Thus, a first field 630 can be manipulated to select a gender, a second field 631 can be manipulated to select an age, a third field 632 can be manipulated to select a height and a fourth field 633 can be manipulated to select a BMI.

Referring now to FIGS. 6A, 6B and 6C, a visual graphical element 635, when selected, is configured to allow the selected and validated one or more reference limits 607 associated with the metadata 609 in the plurality of data records 615 to be automatically exported to the diagnostic/monitoring system 102. The validated and selected one or more of the plurality of data records 615 are indicated by associated checked boxes 640.

Referring back to FIG. 1C, at step 132, the RLG module 110 is configured to receive electrodiagnostic or neuromonitoring data indicative of a second patient test. At step 134, the RLG module 110 is configured to apply at least one diagnostic analysis to the data indicative of the second patient test using the one or more reference limits.

FIG. 1D shows a flowchart of a plurality of steps of a method 100d of applying statistical methods and principles in order to compile the one or more reference limits, in accordance with some embodiments of the present specification. In embodiments, the method is implemented by the RLG module 110 in the closed loop RLG system 100 of FIG. 1A with reference to the method 100d of FIG. 1D. In various embodiments, for each of age group of a plurality of age groups (such as, for example, children, adults, elderly), for each type of test from a plurality of test type (such as, for example, motor, sensory), for each combination of anatomies from a plurality of anatomies (such as, for example, all recorded nerves, stimulation, and recording positions), and for each test parameter from a plurality of test parameters (such as, for example, latency, amplitude), the method 100d implements the following steps:

At step 150, the RLG module 110 is configured such that the module determines the largest combination of predictors (for example, age and height, age, height and gender) that results in at least a minimum number of test parameter values.

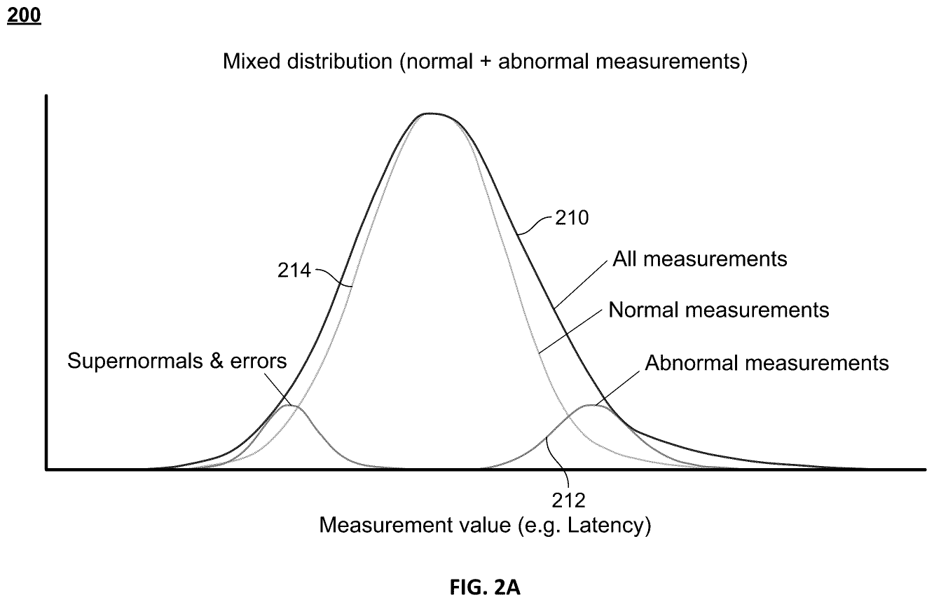

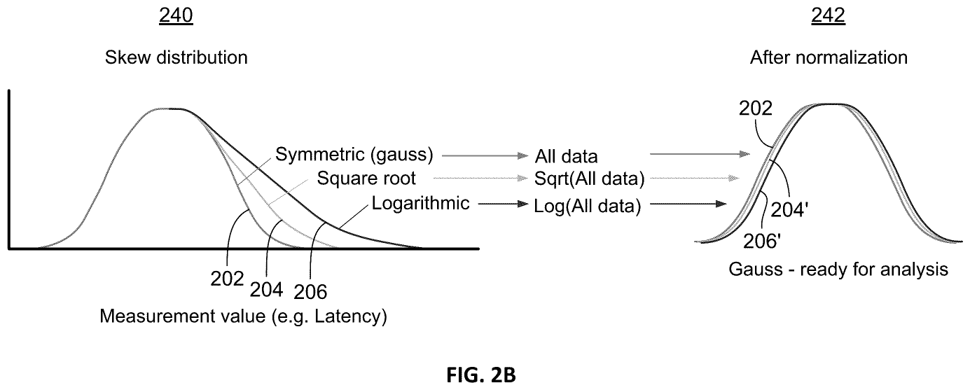

At step 152, the RLG module 110 is configured such that the module extracts data indicative of normal reference values from a mixed clinical dataset obtained in a clinical routine environment from a plurality of separate patient tests. Thus, the data, in embodiments, refers to test results/values that are extracted and then analyzed to determine which values should be used in the creation of reference limits. In some embodiments, the minimum number of test parameter values is 500 and the maximum number of test parameter values corresponds to the last 5000 (sorted by the test date). The data extraction is performed automatically, by the RLG module 110, without any manual editing of data and normal reference limits are automatically constructed based on the extracted normal reference values for each parameter and anatomy. As shown in FIG. 2A, a mixed distribution 210 shows all obtained data for one parameter (for example, latency), a second distribution 212 is indicative of abnormal, supernormal (i.e. extreme normals) and erroneous data (for example, bad markers placement, test recordings etc.), and a third distribution 214 is indicative of normal reference values that need to be extracted to create normal reference limits.

At step 154, the RLG module 110 is configured such that the module generates a histogram using the extracted data. At step 156, the RLG module 110 is configured such that the module applies an optimal normalization method to the extracted data to minimize skewness of distribution of the extracted data. If a data distribution is skewed, normalization is required to obtain a symmetrical Gaussian distribution before further statistical operations are applied. FIG. 2B, in a first view 240, shows a symmetric distribution 202 of test data for a test parameter (such as, latency), a first skewed distribution 204 of test data for the test parameter and a second skewed distribution 206 of test data for the test parameter. Depending on a degree of skewness, an optimal normalization method is selected and applied automatically. For example, no normalization is applied to the symmetric distribution 202, the first skewed distribution 204 is best normalized using a square root transformation and the second skewed distribution 206 is best normalized using a logarithmic transformation. In embodiments, the normalization method selected is that which best achieves a normalized data set (standard bell curve). The second view 242 shows the distributions 202 along with the first normalized distribution 204′ and the second normalized distribution 206′.

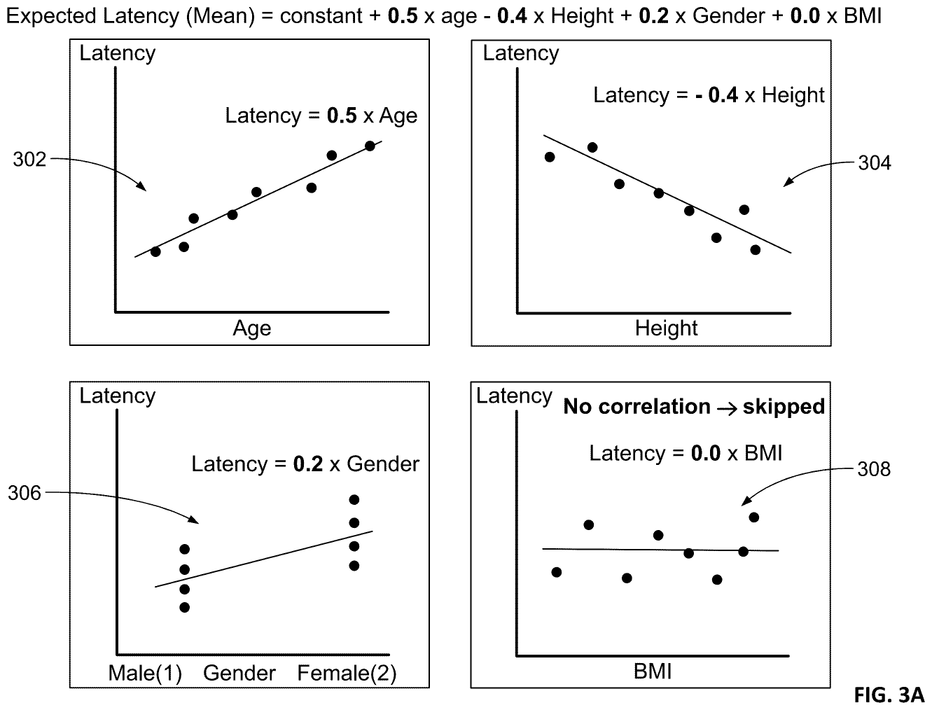

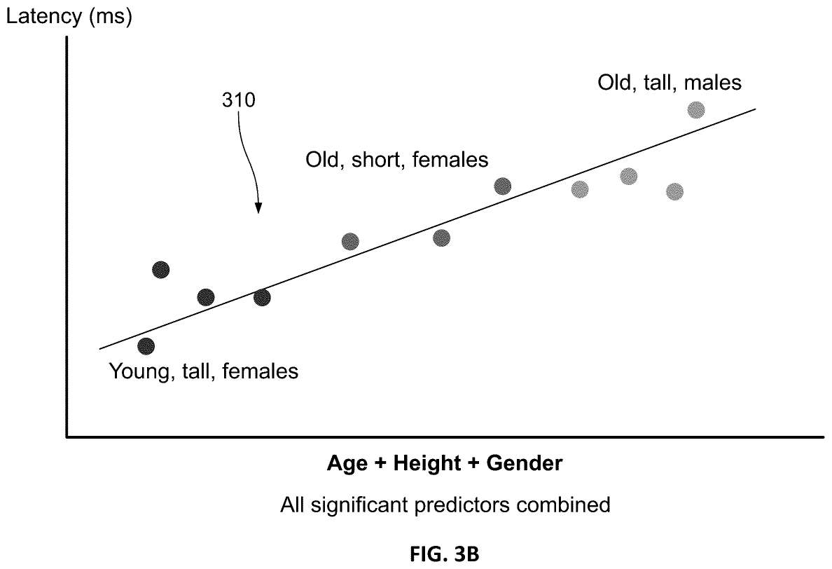

At step 158, after normalization, the RLG module 110 is configured such that the module performs multiple regression analysis on the extracted data. A multiple regression analysis calculates how much impact multiple independent variables (or predictors) have on a dependent variable. For example, a multiple regression analysis may be used to determine the impact that age and height have on latency. In the example shown in FIG. 3A, a first regression model 302 is indicative of an impact of age (independent variable) on latency (dependent variable), a second regression model 304 is indicative of an impact of height (independent variable) on latency (dependent variable), a third regression model 306 is indicative of an impact of gender (independent variable) on latency (dependent variable) and a fourth regression model 308 is indicative of an impact of BMI (independent variable) on latency (dependent variable). Predictors with low impact or no correlation are excluded (such as, BMI in the example). FIG. 3B is a combined plot 310 showing that the normal mean varies depending on the combination of selected predictors (that is, a combination of age, height and gender). For example, a short latency may be normal for a tall young female, while it's abnormal for an elderly tall male.

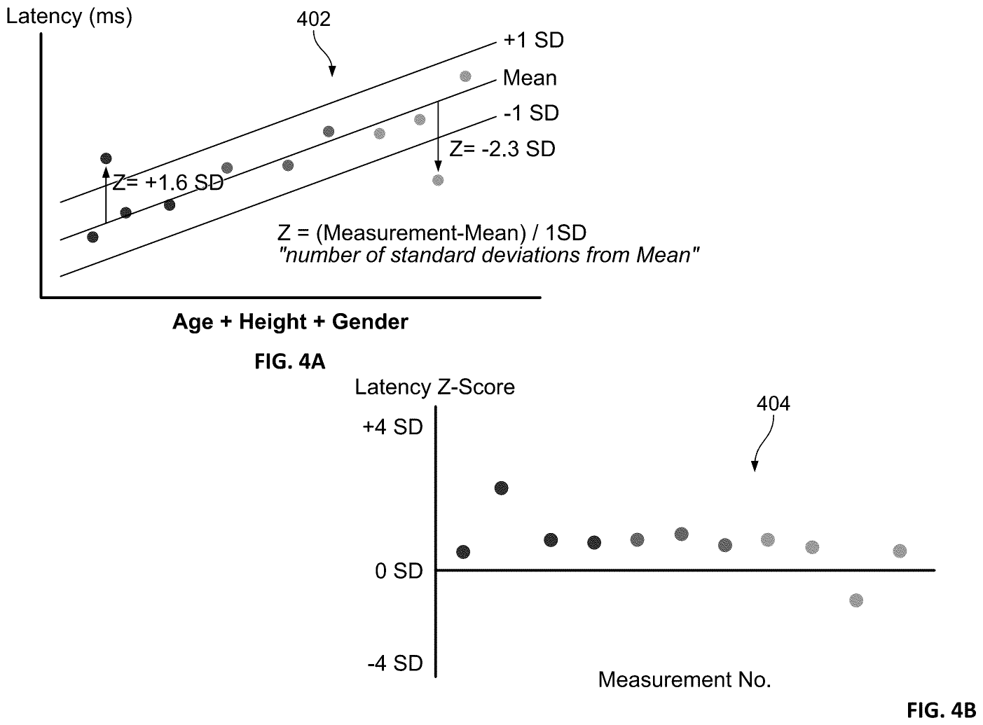

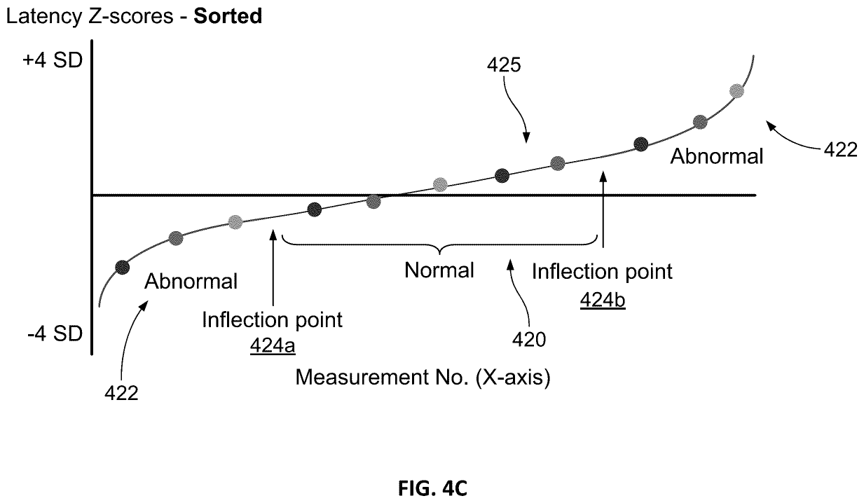

At step 160, the RLG module 110 is configured such that the module calculates a Z-score for each test parameter value and thereafter sorts and smooths the calculated Z-scores. Referring to a first plot 402, shown in FIG. 4A, and a second plot 404, shown in FIG. 4B, to make measurements comparable, a transformation from measured test parameter values to Z-scores is performed. In a multiple regression analysis, the standard deviation (SD) is calculated, which is a measure of the closeness of values to the mean. The Z-score is the distance, in SD, from the expected mean value (center line 402 in the first plot 400a of FIG. 4A). The Z-score transformation removes the predictor generated slope of the mean, thus making the values from different patients comparable.

At step 162, the RLG module 110 is configured such that the module plots Z-scores versus measurement number in a CDF (cumulative distribution function) plot. At step 164, the RLG module 110 is configured such that the module calculates a slope of the center part of the CDF plot (where, midpoint is determined as ±15% of data, for example). At step 166, the RLG module 110 is configured such that the module generates a ‘normal zone’ as ±4 standard deviations, for example, around a center regression line. At step 168, the RLG module 110 is configured such that the module determines inflection points where the CDF plot first enters the normal zone, searching from the start and end of the CDF plot.

The purpose of the cumulative distribution function plot is to separate abnormal from normal values. As shown in FIG. 4C, the Z-scores are sorted by value and plotted versus measurement number. The center section 420 (or the center regression line) is indicative of the normal values, i.e. lowest Z-score values, while the abnormal values have higher Z-scores, positive or negative, and seen in the outer sections 422 of the CDF plot 425. The separation of normal from abnormal values are defined at a first inflection point 424a and a second inflection point 424b on the CDF plot 425, i.e. where the CDF plot 425 bends away from the center section 420 (or the center regression line).

The normal values to be used for the final calculation of the reference limits model reside between the first inflection point 424a and the second inflection point 424b in the CDF plot 425. The final calculation is based on the actual measurement values and not the Z-Scores. Therefore, the measured values corresponding to the normal Z-scores are extracted. Creation of the CDF plot 425 and extraction of normal values is evaluated for each anatomy and each parameter, i.e. normal values from a patient are used even though other parameters for the same patient may be abnormal. In the same way, a measurement from one side may be abnormal and not used, while the other side is normal and thus used.

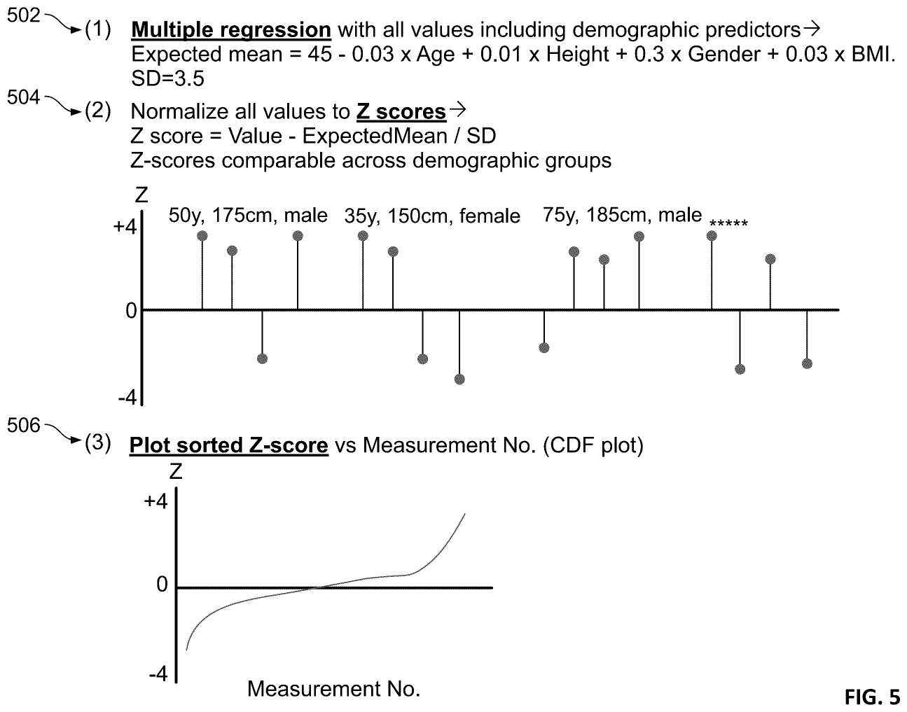

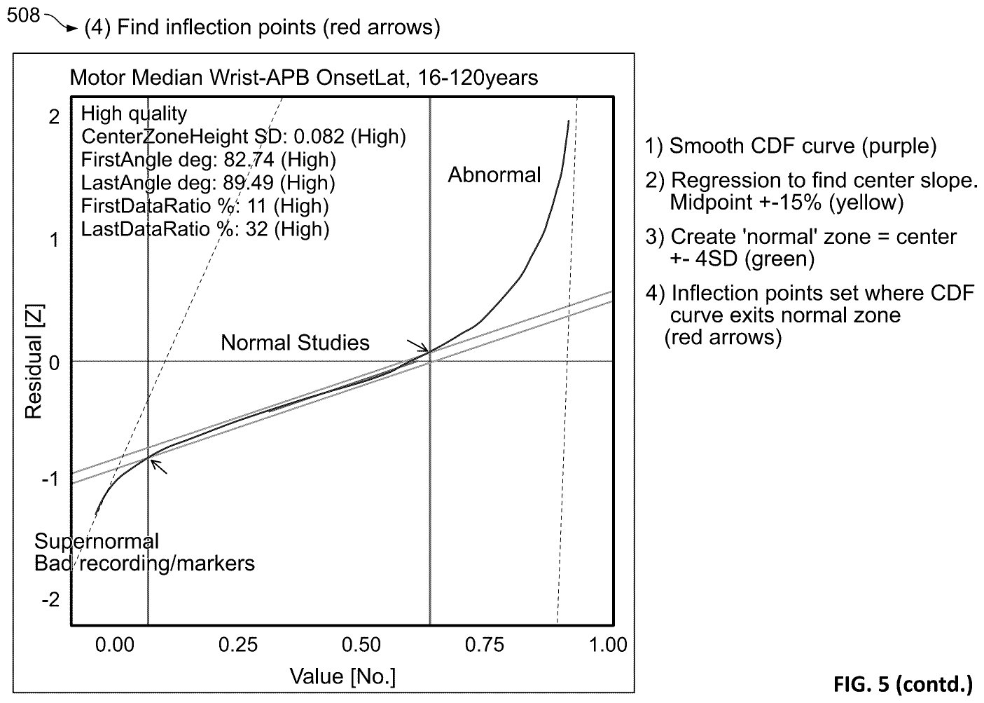

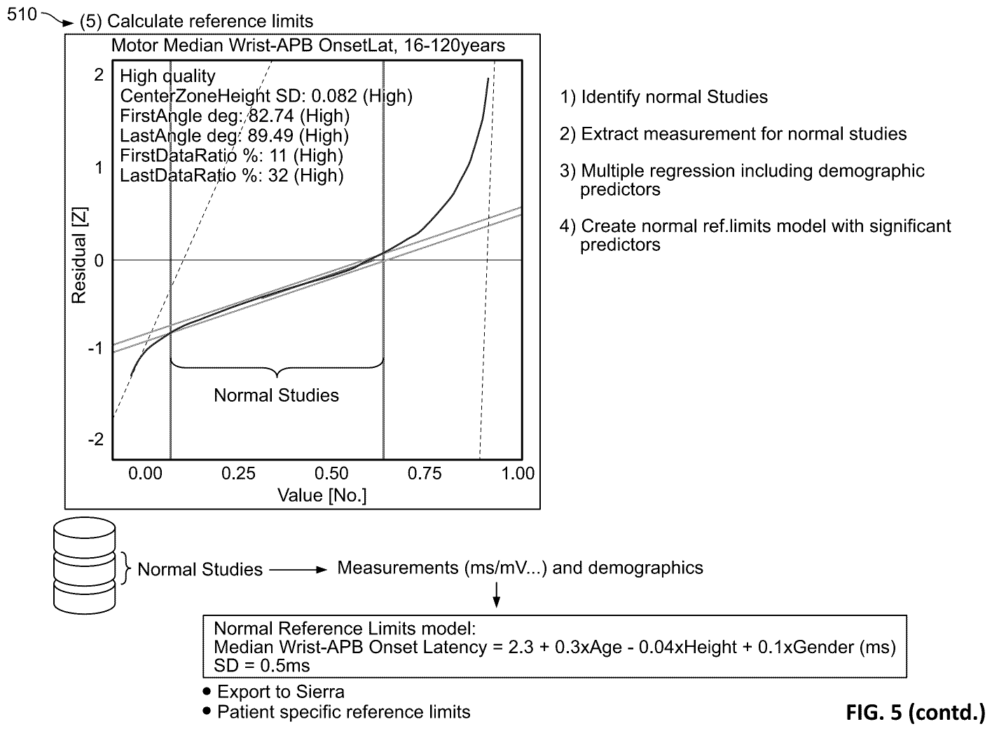

Thus, at step 170, the RLG module 110 is configured such that the module checks quality by calculating the slope of the parts outside the inflection points and number of normal values. At step 172, the RLG module 110 is configured such that the module extracts all values corresponding to the Z-scores between the inflection points. Thus, as shown in FIG. 5, the final normal reference limits model is created, by the RLG module 110 which is configured to perform the steps as follows: at step 502, perform multiple regression with all values including demographic predictors, at step 504, normalize all values to Z scores, at step 506, plot sorted Z-score versus measurement number (to generate a CDF plot), at step 508, find inflection points and at step 510, calculate reference limits.

At step 174, the RLG module 110 is configured such that the module normalizes the extracted distribution if necessary, that is, test for no requirement of normalization, the need for logarithmic transformation, or the need for square root transformation and select the one resulting in least skewness. Thereafter, at step 176, the RLG module 110 is configured such that the module removes predictors with less than 1% impact on the median. At step 178, if any predictor is removed, the RLG module 110 is configured such that the module creates a new multiple regression model and repeat the predictor test. Finally, at step 180, if the final distribution is severely skewed, the RLG module 110 is configured such that the module creates a 5% to 95% range instead of multiple regression model, otherwise perform multiple regression to obtain the normal reference limits model including SD (standard deviation).

In embodiments, steps 150 through 180 are repeated for next parameter, anatomy, test type, age group.

The above examples are merely illustrative of the many applications of the systems and methods of the present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A method of electrodiagnosis or neuromonitoring, comprising:
selecting a electrodiagnostic or neuromonitoring device;
applying at least one electrode to the patient, wherein the at least one electrode is in electrical communication with the electrodiagnostic or neuromonitoring device and wherein the at least one electrode is adapted to detect an electrical signal and transmit it to the electrodiagnostic or neuromonitoring device;
acquiring data indicative of the patient's muscles and/or nerves based on the detected electrical signal;
acquiring a reference limit, wherein the reference limit is generated by:
receiving a selection of at least one of a plurality variables;
acquiring electrodiagnostic or neuromonitoring data based on said selection of at least one of a plurality of variables, wherein the electrodiagnostic or neuromonitoring data was generated from a plurality of separate electrodiagnostic or neuromonitoring tests and wherein the electrodiagnostic or neuromonitoring data is associated with a plurality of variables; and
applying one or more functions to the electrodiagnostic or neuromonitoring data to generate said reference limit;
evaluating said data indicative of the patient's muscles and/or nerves based on the acquired reference signal; and
causing a display of said evaluated data on a display.

2. The method of claim 1, wherein said plurality of variables comprise age, height, weight, gender, and condition.

3. The method of claim 1, wherein at least a portion of the plurality of variables is stored as metadata in relation to the electrodiagnostic or neuromonitoring data.

4. The method of claim 3, wherein the display is further configured to display an option to select one or more portions of the electrodiagnostic or neuromonitoring data and to display said selected one or more portions of the electrodiagnostic or neuromonitoring data with associated metadata.

5. The method of claim 1, wherein said acquisition of the reference limit is further generated by displaying the electrodiagnostic or neuromonitoring data to a user prior to applying the one or more functions.

6. The method of claim 5, further comprising receiving from the user a selection of a portion of the electrodiagnostic or neuromonitoring data.

7. The method of claim 1, further comprising including said data indicative of the patient's muscles and/or nerves in the electrodiagnostic or neuromonitoring data.

8. The method of claim 1, wherein the one or more functions comprises extracting a portion of the electrodiagnostic or neuromonitoring data and generating a histogram.

9. The method of claim 8, wherein the one or more functions further comprises extracting a portion of the electrodiagnostic or neuromonitoring data and applying a normalization process to minimize a skewness of a distribution of the extracted portion of the electrodiagnostic or neuromonitoring data.

10. The method of claim 9, wherein the one or more functions further comprises, after applying said normalization process, applying a regression analysis on said normalized extracted portion of the electrodiagnostic or neuromonitoring data.

11. The method of claim 10, wherein the one or more functions further comprises, after applying said regression analysis, determining Z scores on said normalized extracted portion of the electrodiagnostic or neuromonitoring data.

12. The method of claim 11, wherein the one or more functions further comprises applying a smoothing function to said Z scores.

13. The method of claim 12, wherein the one or more functions further comprises generating a cumulative distribution function plot using said smoothed Z scores.

14. The method of claim 13, wherein the one or more functions further comprises determining a slope of a portion of the cumulative distribution function plot.

15. The method of claim 13, wherein the one or more functions further comprises determining a normal zone of the cumulative distribution function plot and identifying at least one inflection point where the cumulative distribution function plot enters the normal zone.

16. The method of claim 15, wherein the one or more functions further comprises determining a slope of a portion of the cumulative distribution function plot.

17. The method of claim 1, wherein the one or more functions comprises extracting a portion of the electrodiagnostic or neuromonitoring data and generating a plot, wherein said plot comprises Z scores derived from the electrodiagnostic or neuromonitoring data.

18. The method of claim 17, wherein the one or more functions comprises determining one or more inflection points in said plot, wherein said one or more inflection points are indicative of boundaries between an abnormal measurement range and a normal measurement range.

19. The method of claim 1, further comprising generating a graphical user interface, wherein said graphical user interface is configured to display a plurality of reference limits and wherein each of said plurality of reference limits is displayed using a set of parameters.

20. The method of claim 1, wherein the set of parameters comprises two or more of degree of quality, age groups, types of test, anatomies, measured test parameters, normal ranges of values for the measured test parameter, and model.

21. The method of claim 20, wherein the degree of quality is color coded, and the measured test parameter comprises conduction velocity.
